(19) United States
(12) Patent Application Publication
(10) Pub. No.: US 2023/0346694 A1
(43) Pub. Date: Nov. 2, 2023

(54) DEVICES FOR TREATMENT OF BLEEDING IN BODY LUMENS OR SPACES, INCLUDING EPISTAXIS, AND METHODS OF MAKING AND USING THE SAME

(71) Applicant: Bio 54, LLC, Chapel Hill, NC (US)

(72) Inventor: Anton Travis MANASCO, Chapel Hill, NC (US)

(73) Assignee: Bio 54, LLC, Chapel Hill, NC (US)

(21) Appl. No.: 18/172,253

(22) Filed: Feb. 21, 2023

Related U.S. Application Data
(63) Continuation of application No. PCT/US2021/046745, filed on Aug. 19, 2021.
(60) Provisional application No. 63/068,032, filed on Aug. 20, 2020.

(51) Int. Cl. A61K 9/00; A61K 31/195; A61F 13/20; A61F 13/84

(52) U.S. Cl. CPC A61K 9/0043; A61K 31/195; A61F 13/2025; A61F 13/8405; A61F 13/2005

(57) ABSTRACT

In some embodiments, a system includes a nasal packing, a delivery device releasably couplable to the nasal packing and configured to deliver the nasal packing into a nasal cavity of a subject, a reservoir configured to contain medication, and a fluid coupling configured such that the medication can flow from the reservoir to the nasal packing via the fluid coupling.

### CROSS-REFERENCE TO RELATED APPLICATIONS

This application is a continuation of International Patent Application No. PCT/US2021/046745, filed Aug. 19, 2021, entitled “Devices for Treatment of Bleeding in Body Lumens or Spaces, Including Epistaxis, and Methods of Making and Using the Same,” which claims priority to U.S. Provisional Patent Application Ser. No. 63/068,032, entitled “Devices for Treatment of Epistaxis and Methods of Making and Using the Same,” filed on Aug. 20, 2020, each of which is incorporated herein by reference in its entirety for all purposes.

### BACKGROUND

A common ailment for hospital emergency rooms and urgent care center visits is epistaxis (bleeding from the nose). Epistaxis occurs somewhat easily due to the location of blood vessels close to the surface in the lining of the nose. Additionally, the location of the nose makes it a frequent target of injury resulting in epistaxis. Dry air from indoor heat, high altitude, and/or climates with low humidity may also cause epistaxis. Sinusitis and upper respiratory infections may further result in epistaxis. Other causes of epistaxis include nasal use of illicit drugs (e.g., cocaine), tumors, vascular disorders (e.g., Osler-Weber-Rendu), coagulopathies, kidney failure, liver failure, and platelet disorders. Risk factors associated with epistaxis include age over 50 years, anticoagulant or antiplatelet use, congestive heart failure, hypertension, diabetes, and alcohol use. Because epistaxis can cause blood from the hemorrhage site to flow into the stomach, a patient may also experience nausea and even vomiting.

Epistaxis is not normally life threatening, but can occupy precious medical resources. Some episodes of epistaxis will resolve with direct pressure to the bilateral nose inferior to the nasal bone for 10-20 minutes but rebleeding rates are high. Patient adherence to proper technique is highly variable. As such, better methods and devices are needed to treat epistaxis while reducing the patent treatment time at hospital emergency rooms and urgent care centers. Epistaxis is a common emergency department (ED) complaint that is most frequently idiopathic in etiology (85%) and anterior in location (90%) and can generally be controlled in the ED (>70%). The estimated annual cost of epistaxis management is $100 million. Epistaxis can be more problematic for persons who do not have normal blood clotting or coagulation, e.g. person with conditions such as hemophilia, or persons whose clotting or coagulation has been medically altered or reduced. Oral vitamin K antagonists (IE warfarin) are an independent risk factor for epistaxis, with an odds ratio of 11.6. One ED-based study found that 61% of patients with epistaxis were on anticoagulants or antiplatelet agents.

It would be useful to further address these and other problems or challenges associated with epistaxis treatment.

In addition to epistaxis, there are many other contexts in which a patient may experience bleeding in a body lumen or space (natural or surgically-created) that would also benefit from availability of improved devices and methods for achieving hemostasis.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

Disclosed herein are devices, methods of using devices, and methods of making devices for treatment of epistaxis. In some embodiments, a system includes a nasal packing, a delivery device releasably couplable to the nasal packing and configured to deliver the nasal packing into a nasal cavity of a subject, a reservoir configured to contain medication, and a fluid coupling configured such that the medication can flow from the reservoir to the nasal packing via the fluid coupling.

Additional features, aspects and/or advantages will be recognized and appreciated upon further review of a detailed description of the illustrative embodiments taken in conjunction with the accompanying drawings.

### BRIEF DESCRIPTION OF THE DRAWINGS

FIG. 1A is a partial cross-sectional view of a head, showing the structures of the mouth, nose, and sinuses.

FIG. 1B is a partial cross-sectional view of a head, showing the vasculature in the nose and sinuses.

FIG. 2A shows top and bottom views of a nasal tampon usable for treatment of epistaxis, and FIG. 2B shows the tampon being inserted into the nose of a patient.

FIG. 3 is a schematic illustration of an epistaxis treatment system, according to an embodiment.

FIG. 4A is a schematic illustration of the epistaxis treatment system of FIG. 3, with packaging removed from the epistaxis treatment device, shown with a portion of the epistaxis treatment device disposed in the nasal cavity of a patient.

FIG. 4B is a schematic illustration of the epistaxis treatment device of FIG. 4A with the delivery device and other components removed, shown with the nasal packing disposed in the nasal cavity of the patient.

FIG. 4C is a schematic illustration of the nasal packing of FIG. 4B, shown expanded to press against nasal tissue.

FIG. 4D is a schematic illustration of the nasal packing of FIG. 4B, shown unexpanded to release pressure against nasal tissue after hemostasis of the nasal tissue has been achieved.

FIG. 4E is a schematic illustration of the nasal packing of FIG. 4B, shown withdrawn from the nasal cavity of the patient.

FIG. 5 is a flow chart of a method of using the epistaxis treatment system of FIGS. 3 and 4A to 4E to treat epistaxis.

FIG. 6 is a schematic illustration of an epistaxis treatment system, according to an embodiment.

FIG. 7 is a schematic illustration of a nasal packing, according to an embodiment.

FIG. 8 is a schematic illustration of a nasal packing and an expander, according to an embodiment.

FIGS. 9 and 10 are schematic illustrations of an epistaxis treatment system in an unexpanded configuration and an expanded configuration, respectively, according to an embodiment.

FIGS. 11 and 12 are schematic illustrations of an epistaxis treatment system in an unexpanded configuration and an expanded configuration, respectively, according to an embodiment.

FIG. 13 is a schematic illustration of a treatment system, according to an embodiment.

FIG. 14 is a schematic illustration of the treatment system of FIG. 13, with packaging removed from the treatment device, shown with a portion of the treatment device disposed in a body cavity of a patient.

### DETAILED DESCRIPTION

The detailed description herein serves to describe non-limiting embodiments or examples involving various inventive concepts and uses reference numbers for ease of understanding these examples. Common reference numbers between the figures refer to common features and structure having the same or similar functions, as will be understood. While various figures will have common reference numbers referring to such common features and structure, for purposes of conciseness, later figure descriptions will not necessarily repeat a discussion of these features and structure.

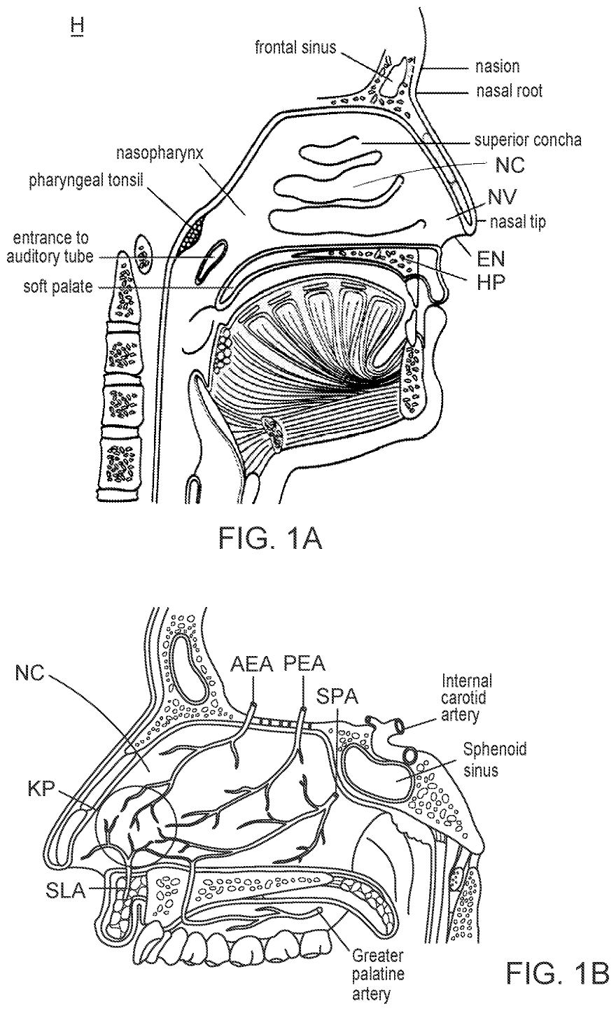

For reference, FIGS. 1A and 1B illustrate a head H of a human, e.g. of a patient to be treated. Head H includes a nose N and defines a nasal cavity NC. The nose N has external nares EN, a hard palate HP, and a nasal vestibule NV. The nasal vestibule NV defines the external opening of the nose N. The vasculature of nose N is shown in more detail in FIG. 1B, and includes the superior labial artery SLA and the anterior ethmoidal artery AEA. Both arteries extend to Kiesselbach's plexus KP, which is a common location for anterior epistaxis. The vast majority of epistaxis cases are arterial. The anterior location is Kiesselbach's plexus KP, while the less frequent posterior cases originate from the sphenopalatine or posterior ethmoidal arteries.

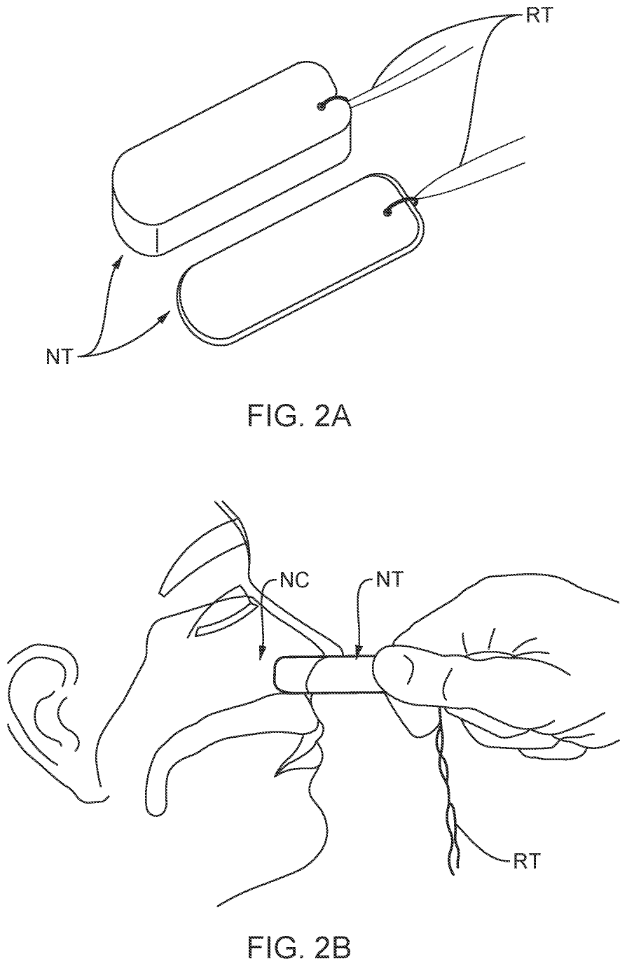

A nasal tampon that can be used to treat epistaxis (e.g., anterior or posterior epistaxis) is shown in FIGS. 2A and 2B. As shown in FIG. 2A, the nasal tampon NT can be formed of an absorbent, expandable material that can be inserted into the nasal cavity NC of a person experiencing epistaxis (e.g., anterior epistaxis). As shown in FIG. 2B, the nasal tampon NT can be inserted into the nasal cavity NC manually, e.g. by a physician or other health care practitioner, or by the patient or a caregiver, then manual pressure can be applied, e.g. against the exterior of the nose, to press nasal membrane, e.g. at Kiesselbach's plexus KP, against the nasal tampon NT. If and when hemostasis is achieved, the nasal tampon NT can be removed from the nasal cavity NC, e.g. by grasping and pulling on the retrieval tether RT.

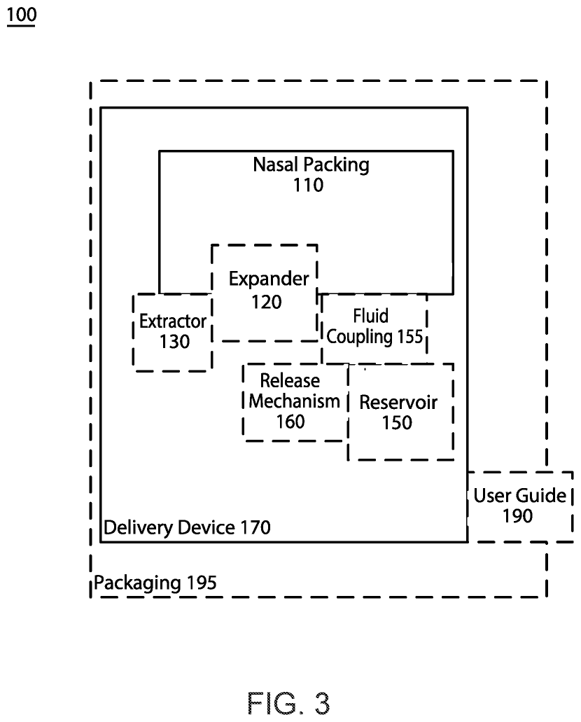

An embodiment of an epistaxis treatment system 100 is illustrated schematically in FIG. 3. As shown in FIG. 3, the epistaxis treatment system 100 includes a nasal packing 110, an optional expander 120 coupled to the nasal packing 110, an optional extractor 130 coupled to nasal packing 110, one or more optional reservoirs 150 fluidically coupled to the nasal packing 110 via a fluid coupling 155 and containing one or more medications, an optional release mechanism 160 coupled to the reservoir 150 and the fluid coupling 155, a delivery device 170 releasably coupleable to the nasal packing 110, an optional user guide 190 associated with the delivery device 170, and optional packaging 195 to contain the other components of the epistaxis treatment system 100.

The nasal packing 110 is sized, configured, and formed of material, suitable for insertion into a nasal cavity NC of a person experiencing epistaxis, where it may absorb blood released from the person's nasal vasculature and/or may apply (or be caused to apply) pressure to target nasal tissue at the site of the bleeding (such as Kiesellbach's plexus KP) to aid in achieving hemostasis. In some embodiments, the nasal packing 110 may be any commercially available nasal packing, nosebleed plugs, etc. The size of nasal packing 110 may be selected to be appropriate for the person to be treated, e.g. may be of different size for infants, children, or adults of different sizes. The nasal packing 110 may be configured in any suitable geometry. For example, the nasal packing 110 may have any cross-sectional shapes (circular, oval, triangular, rectangular, etc.). The cross-sectional shape and/or size of the nasal packing 110 may be constant along a length of the nasal packing 110, or may vary (e.g. the same cross-sectional shape but varying size, i.e. tapered, and/or differing cross-sectional shapes (e.g. circular at the distal end to be inserted into the nasal cavity NC but rectangular at a proximal end)), as described in more detail for specific embodiments described below.

The nasal packing 110 may be formed of any one or more materials having suitable physical properties. For example, the material is preferably capable of absorbing blood, which may avoid the aspiration of blood by the treated patient. Desirably it will also swell or expand upon absorbing fluid such as blood. It is preferably also capable of containing, absorbing, wicking, or otherwise transporting one or more medications for application to target nasal tissue in the nasal cavity NC. It should be biologically compatible with the nasal tissue. Preferably the one or more materials are also non-reactive, or otherwise compatible with any medication(s) to be transported by the nasal packing 110, e.g. not alter the composition, delivery, or efficacy of the medication(s) or degrade or otherwise lose any of its desired physical properties upon exposure to the medication(s) over the maximum duration of epistaxis treatment. The material is preferably sufficiently stiff, resilient, etc. to be capable of applying a sufficient amount of pressure against nasal tissue to aid in achieving hemostasis, i.e. function as a hemostat. It may have surface properties that facilitate insertion into, and removal from, nasal cavity NC, e.g. it may have a smooth and relatively “slippery” surface (which may be enhanced by absorption of blood or other fluid, e.g. medication(s)). Preferably the material may be compressed so that the nasal packing 110 has a relatively smaller cross section for insertion into the nasal cavity NC (and/or containment within delivery device 170), and may then be expanded (e.g. by application of mechanical force from the expander 120) and/or self-expand (e.g. by its own resilience and/or by absorbing blood, medication(s), and/or other fluid(s)).

Suitable materials for nasal packing 110 include polymers or other compositions, such as polyvinyl alcohol (PVA), polyurethane (hydrophilic or otherwise), polypropylene, which may be formed into foams (open or closed cell) (e.g., a porous expandable foam), natural fibers such as cotton, linen, wool, etc. in woven or non-woven (e.g. felt) form, and/or layered matrices of foam and/or gauze packing. The nasal packing 110 may be formed monolithically of a single material, or may be formed as a composite or other aggregation of different materials. For example, the nasal packing 110 may be primarily formed of one material, and have a relatively thin covering of a second material. The materials may achieve the desired functions in different ways. For example, absorption of blood may be achieved mechanically, e.g. by capillary wicking, and/or chemically, such as by absorption into, for example, molecular sieves or other desiccants, or combination with materials such as clays, e.g. kaolin, bentonite, montmorillonite, saponite, polygorskite, attapulgite, and/or sepiolite. In some embodiments, clay may be dispersed in a liquid medium.

As explained in more detail below, the nasal packing 110 may include, incorporate, or embody a reservoir for medication(s), and a different material may be used to form or bound such a reservoir. In some embodiments, the nasal packing 110 can define or include internal structures or passages to aid flow of medication through the nasal packing 110 and/or preferentially direct flow toward target nasal tissue.

The expander 120 may be included in the epistaxis treatment system 100 to provide an expansion force to the nasal packing 110, e.g. to increase the pressure that the nasal packing 110 may apply to target nasal tissue during use, to enhance hemostasis. The expander 120 may be implemented in a variety of ways, including those described in specific embodiments below. For example, the expander 120 may be disposed internally to the nasal packing 110, and be selectively expandable or deployable in a lateral/radial and/or longitudinal/axial direction, thus increasing the size or extent of the nasal packing 110 in such direction(s). Alternatively, the expander 120 may be disposed on one side of the nasal packing 110, opposite to the side to be pressed against the target (e.g., bleeding) nasal tissue, and urge the nasal packing 110 laterally when expanded or deployed. The expander 120 is preferably configured to be expandable over a sufficient distance to urge the relevant portion of the nasal packing 110 into contact with nasal tissue (if not already in contact) and to provide a sufficient hemostatic force against the target nasal tissue (e.g. to generate sufficient pressure given the stiffness or modulus of the material(s) of which nasal packing 110 is formed). The expander 120 may be self-expanding, e.g. formed of a resilient material and held in a compressed state until expansion is desired, and then released from its compressed state, e.g. upon discharge from the delivery device 170. For example, the expander 120 may be formed from a resilient material and configured so that it can be compressed and then self-expand, e.g. as a cylindrical stent, a coil spring, u-shaped spring, etc. Alternatively, expander 120 may require external actuation to cause it to expand. For example, expander 120 may be implemented as a balloon that may be inflated by a fluid (gas or liquid) from a delivery configuration to an expanded, use configuration, and the inflation fluid may be delivered to the balloon from a source of pressurized fluid controlled by a user (e.g., a bladder optionally coupled to a manometer) via a tubular member defining a lumen. Optionally, the expander 120 may be configured to be unexpanded, compressed, or otherwise reduced in size in the relevant direction(s) to allow, or cause, nasal packing 110 to reduce in size, e.g. at the completion of treatment to ease the withdraw of nasal packing 100 from the nasal cavity NC.

The extractor 130 may be included in the epistaxis treatment system 100 to enable or assist in removal of the nasal packing 110 from the nasal cavity NC at the completion of treatment. The extractor 130 may be a separate structure and/or material from the nasal packing 110, and/or may be a part of the nasal packing 100. For example, the extractor 130 may be a suture or other elongate tether (e.g., similar to or the same as the retrieval tether RT shown in FIGS. 2A and 2B) fixed at one end to the nasal packing 110 and having a free end that can be grasped by a user and configured to transmit to the nasal packing 110, and sustain, sufficient force applied by the user to withdraw the nasal packing 110 from the nasal cavity NC after treatment, when the nasal packing 110 may be in an expanded state (e.g. by application of force/displacement from the expander 120 and/or by absorption of blood, medication(s), and/or other fluid(s)). In other embodiments, the extractor 130 may be a suitably sized/shaped proximal portion of the nasal packing 110, configured to be grasped by the user and sustain the required extraction force.

The epistaxis treatment system 100 is preferably configured to deliver one or more medications to target nasal tissue during treatment, e.g. via the nasal packing 110. The medication(s) may be contained in one or more reservoirs 150 (which, as noted above, may be separate from or incorporated into nasal packing 110), may be selectively released from the reservoir(s) 150 by one or more release mechanisms 160, and may be conveyed from the reservoir(s) 150 to the nasal packing 110 by one or more fluid couplings 155. For example, the reservoir 150 may be implemented as a container (ampoule or the like) having a volume sufficient to contain a therapeutically effective amount of the medication and formed of material impermeable to, and non-reactive with, the medication or its constituents, such as glass, metal, plastic, polymer, etc. The reservoir 150 may have an opening through which the medication can be introduced into the reservoir 150 and/or selective selectively released therefrom.

The reservoir 150 may be contained, in whole or in part, within the nasal packing 110, or may be coupled thereto via a fluid coupling 155, such as a tube, wick, etc. The medication may be selectively released from the reservoir 150 so that the medication can be received in the nasal packing 150, e.g. by the release mechanism 160. The release mechanism 160 may be, for example a valve, which may be opened to establish fluidic communication between the reservoir 150 and the nasal packing 110, directly or via fluid coupling 155, and may also be selectively closed to fluidically isolate the medication in the reservoir 150. The release mechanism may be a removable or frangible cap or other closure closing an opening in reservoir 150. The reservoir 150 may itself be frangible, e.g. formed of glass that may be readily broken or at least partially of a material that bursts when the contents of the reservoir 150 are above a threshold pressure, and the medication can be released from the reservoir 150 by causing or allowing the reservoir 150 to be broken. As noted above, in some embodiments the reservoir 150 may be a part of the nasal packing 110. For example, the material of the nasal packing 100 may be soaked or saturated with one or more medications, and enclosed with a film or other thin layer of material impermeable to and non-reactive with the medication(s). The medication(s) can be released from the reservoir 150 by removing the film or other thin layer of material, exposing the surface of the nasal packing 110 so that the medication(s) can be delivered to the nasal tissue with which the surface of the nasal packing 110 is placed in contact.

The delivery device 170 may be any suitable device manipulable by a user to dispose the nasal packing 110 into a desired location, e.g. in nasal cavity NC, and to deposit the nasal packing 110 in the desired location. For example, the delivery device 170 may be a mechanical syringe, such as used with the Rhino Rocket Nasal Packing available from Summit Medical, that includes a “barrel” in which the nasal packing 110 may be disposed (e.g. in a compressed state) and having a distal end suitable for insertion into the nasal cavity NC, and a proximal end that may be grasped by the user, and a “plunger” movable relative to the barrel and to which a user can apply distally directed force to engage the distal end of the plunger with the nasal packing 100 and urge it distally through the barrel and out of a distal end of the barrel. In some embodiments, the reservoir 150, the release mechanism 160, and/or the fluid coupling 155 can be disposed within the barrel (e.g., between the plunger and the nasal packing 110). In some embodiments, the delivery device 170 can include a filter between the reservoir 150 and the nasal packing 110 to prevent unwanted material (e.g., particles above a certain size and/or glass pieces) from reaching the nasal packing 110. In other embodiments, the delivery device 170 may not contain nasal packing 110, but instead just provide a handle by which the user may hold the nasal packing 110 to be able to insert it into nasal cavity NC, and selectively release the nasal packing 110.

The epistaxis treatment system 100 may include a user guide 190. The user guide 190 may include instructions for operation of the epistaxis treatment system 100 to treat a person experiencing epistaxis. The instructions may be in the form of textual and/or graphical information, which may be presented on fixed substrate (e.g. paper) or on a display (e.g. screen), and/or may use other sensory modalities, including audible (spoken instructions) and/or tactile (haptic feedback to the user). The user guide 190 may be disposed on (e.g. printed on) or coupled to (e.g. mechanically attached) any one or more component(s) of the epistaxis treatment system 100, including the delivery device 170, the packaging 190, the nasal packing 110, and/or the reservoir 150. The user guide 190 may be separate from any of the components of the epistaxis treatment system 100, but may be associated therewith, e.g. disposed in the packaging 195 along with the other components of the epistaxis treatment system 100. In some embodiments, the user guide 190 may be implemented in whole or in part in software usable on a device such as smart phone, e.g. may be the form of an “app” that can be downloaded onto the smart phone and launched by a user in preparation for using the epistaxis treatment system 100.

The packaging 195 may be implemented in the same manner as any known medical device packaging, to contain the other components of the epistaxis treatment system 100, to protect the components from the environment, and optionally to preserve sterility of the components. The packaging 195 is preferably configured to be readily opened by a user, e.g. by peeling a cover from a tray, when the user desires to access and use epistaxis treatment system 100. The packaging 195 may be implemented in many other ways, including for example a bag or box.

The medication(s) described herein may be any medication that would be desirable to deliver to the patient experiencing epistaxis, preparatory to or as part of treatment of the epistaxis. Categories of medications M may include vasoconstrictors, antifibrinolytics, antibiotics, recombinant clotting factor medications or any combination thereof. A vasoconstrictor may be useful to help bleeding vessels constrict prior to administration of a hemostatic medication such as an antifibrinolytic, and may desirably be delivered to the nasal tissue at or around the site of the bleeding before, during, and/or after application of nasal packing 110 to the nasal tissue. Suitable vasoconstrictors may include phenylephrine, oxymetazoline (Afrin), and epinephrine. An antifibrinolytic agent may be useful to stabilize the blood clot, and may also desirably be delivered to the nasal tissue at or around the site of the bleeding before, during, and/or after application of nasal packing 110 to the nasal tissue. Suitable antifibrinolytics may include aminocaproic acid, tranexamic acid (TXA), aprotinin, protaaminomethylbenzoic acid, and fibrinogen. Protamine, a reversal agent for the anticoagulant heparin could be used before, during, or after use of the above medications. As noted above, the epistaxis treatment system 100 and medication(s) M may be particularly helpful for treatment of patients who are susceptible to bleeding or for whom it may be difficult to achieve hemostasis, such as patients who are taking anticoagulation and/or antiplatelet medications. Representative anticoagulation medications include warfarin, heparin, and low-molecular weight heparin, dabigatran, argatorban, hirudin, rivaroxaban, apixaban, edoxaban, fondaparinux, and bivalirudin. Representative antiplatelet medications include aspirin (acetylsalicylic acid or ASA), cangrelor, ticagrelor, lopidogrel, prasugrel, epifibatide, tirofiban, and abciximab. Other patients for whom this treatment system (100) and medications (M) could be helpful include patients with vascular disorders (e.g., Osler-Weber-Rendu), coagulopathies, kidney failure, liver failure, bone marrow suppression (pathologically or medication-induced) or platelet disorders.

In some embodiments, the medication can include a pharmaceutical composition including a therapeutically effective amount of TXA, one or more antibiotic(s), one or more anesthetic(s), one or more non-steroid anti-inflammatory drug(s), and/or an excipient or carrier that facilitates local administration. For example, in some embodiments, the therapeutically effective amount of tranexamic acid is between 1-70% by weight of the composition. In some embodiments, the one or more antibiotic(s) can include sulfacetamide, mupirocin, erythromycin, sulfadiazine, mafenide, tetracycline, bacitracin, neomycin, and polymyxin B. In some embodiments, the one or more antibiotic(s) can include bacitracin, neomycin, and polymyxin B. In some embodiments, the excipient or carrier permits the composition to remain in contact with a bleeding wound. In some embodiments, the excipient or carrier comprises an ointment, a cream, a liniment, a paste, a patch, a lotion, a gel, a shampoo, a hydrogel, a liposome, a spray, an aerosol, a solution, a sponge, a film, a plaster, a surgical dressing, a bandage, or an emulsion. In some embodiments, the excipient or carrier permits instillation of the composition, wherein the instillation is selected from nasal instillation, rectal instillation, and bladder instillation. In some embodiments, the one or more anesthetic(s) can include lidocaine, proparacaine, procaine, tetracaine and combinations thereof. In some embodiments, the one or more non-steroid anti-inflammatory drug(s) can include ketorolac, ketoprofen, flurbiprofen, bromfenac, diclofenac and/or combinations thereof.

In some embodiments, the medication can include analgesics, including but not limited to, opiates such as codeine, morphine, oxycodone, etc.; acetaminophen; anti-inflammatory agents, including nonsteroidal anti-inflammatory drugs, aspirin, etc.; antibiotics or another antimicrobial drugs or compounds; antihistamines (e.g., cimetidine, chloropheniramine maleate, diphenhydramine hydrochloride, and promethazine hydrochloride); antifungal agents; ascorbic acid; rutin; thrombin; botanical agents; etc.; and combinations thereof. The medication can also include magnesium sulfate, sodium metaphosphate, calcium chloride, dextrin, and combinations thereof.

In some embodiments, the medication can include sterile water and/or normal saline. In some embodiments, the medication can include between about 50% and about 90% tranexamic acid and between about 10% and about 50% sterile water or normal saline. In some embodiments, the medication can include at least one of a liquid and a gel. In some embodiments, the medication can have a viscosity between about 0.75 millipascal-seconds and about 0.98 millipascal-seconds at about +25 degrees Celsius.

In some embodiments, the reservoir(s) 150 and/or release mechanism 160 are configured such that a metered dose can be provided from the reservoir (e.g., to the nasal packing 195 and/or to the patient). In some embodiments, the metered dose can be between about 1.5 milliliters and about 2.5 milliliters. In some embodiments, the metered dose can be between about 2.5 milliliters and about 4.5 milliliters. In some embodiments, the reservoir(s) 150 can be configured to contain any suitable amount of medication, such as between about 3 milliliters and about 7 milliliters of medication.

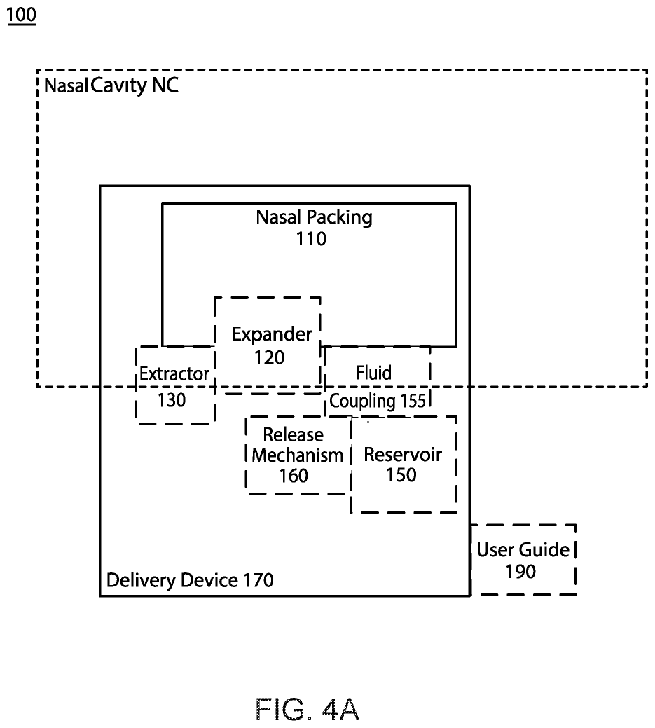

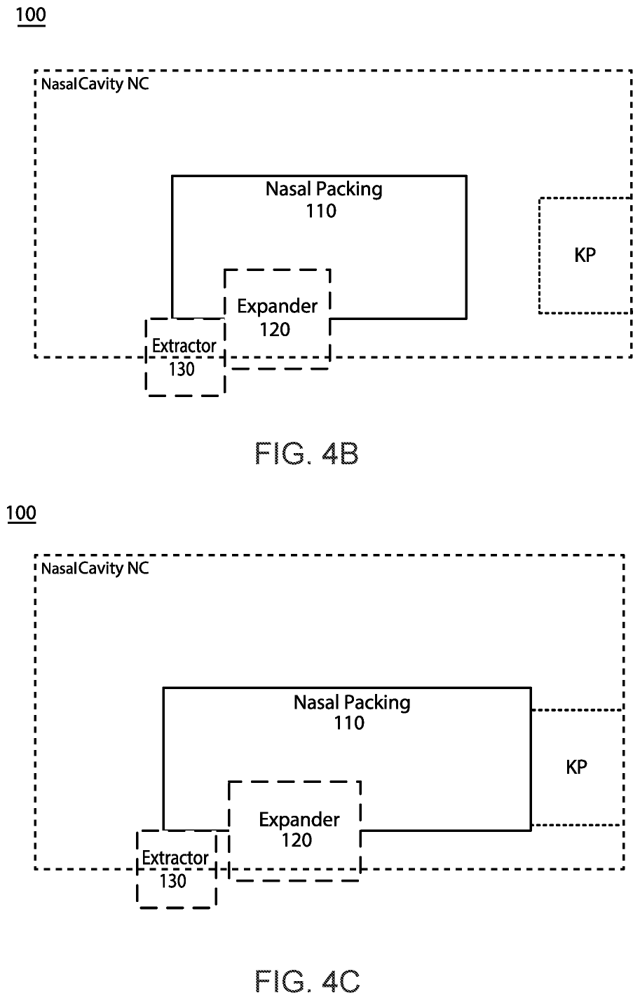

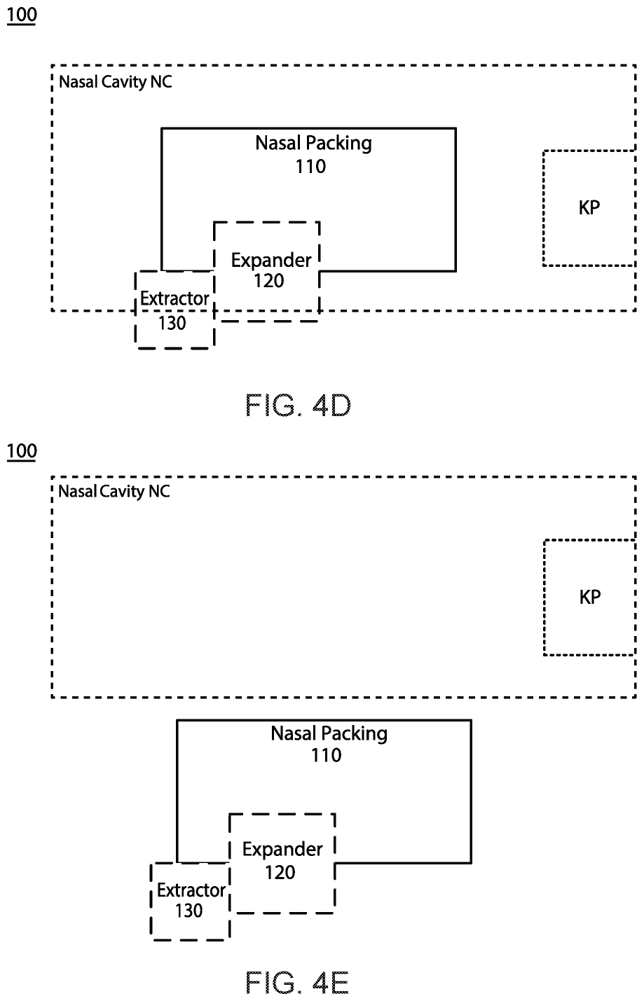

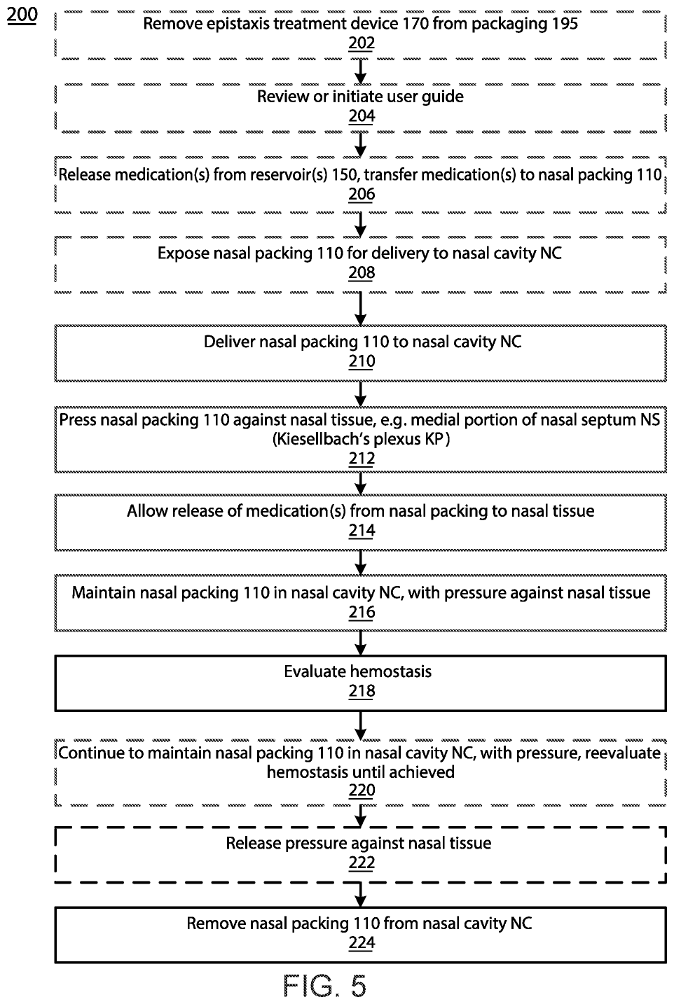

A method of using epistaxis treatment system 100 to treat epistaxis is shown in FIG. 5 and illustrated with reference to FIGS. 4B to 4E. As shown in FIG. 5, in some embodiments, the epistaxis treatment device 170 can be removed, at 202, from the packaging 195. The user guide 190 can optionally be reviewed or initiated, at 204. At 206, optionally, medication(s) can be released from the reservoir(s) 150 and transferred to the nasal packing 110. The nasal packing 110 can be exposed, at 208, for delivery to the nasal cavity NC (e.g., before or after the nasal packing 110 has been disposed within the nasal cavity NC).

As shown in FIG. 5 and illustrated in FIG. 4A, the nasal packing 110 can be delivered, at 210, to the nasal cavity NC. As shown in FIG. 4A and referenced above, in some embodiments, the nasal packing 110 can be delivered to the nasal cavity NC within, extending from, or coupled to the delivery device 170. After the nasal packing 110 has been exposed and disposed within the nasal cavity NC, the delivery device 170 can be separated from the nasal packing 110. The delivery device 170 can be removed from the nasal cavity, leaving the nasal packing 110 disposed within the nasal cavity NC as illustrated in FIG. 4B.

The nasal packing 110 can be pressed, at 212, against the nasal tissue. For example, as illustrated in FIG. 4C, the nasal packing 110 can be pressed against a medial portion of the nasal septum NS (e.g., Kiesellbach's plexus KP). In some embodiments, as shown in FIG. 4C, the nasal packing 110 can be pressed against the nasal tissue via expanding the nasal packing 110 with the expander 120. As described above, alternatively or additionally, in some embodiments, the nasal packing 110 can be pressed against the nasal tissue via the nasal packing 110 self-expanding and/or via applying pressure to an exterior portion of the nose (e.g., with fingers).

Medication(s) can be allowed to be released, at 214, from the nasal packing 110 to the nasal tissue. The nasal packing 110 can be maintained, at 216, in a nasal cavity NC with pressure applied by the nasal packing 110 against the nasal tissue. At 218, hemostasis can be evaluated. For example, a hemostasis condition of the nasal tissue can be evaluated to determine if the hemostasis condition meets a target hemostasis condition. The hemostasis condition of the nasal tissue can be evaluated via any suitable method. For example, the nasopharynx of the patient can be evaluated by looking down the throat of the patient to evaluate whether fluid or blood is flowing from the target nasal tissue beyond the user's nasopharynx. Alternatively, after a preset period of time (e.g., ten to thirty minutes), the nasal packing 110 can be removed and checked to determine whether or not hemostasis has been achieved. At 224, nasal packing 110 can be removed from the nasal cavity NC.

In some embodiments, after evaluating hemostasis at 218, the nasal packing 110 can continue to be monitored, at 220, in the nasal cavity NC while continuing to apply pressure to the nasal tissue and hemostasis can be reevaluated until achieved. For example, if the hemostasis condition of the nasal tissue fails to meet a target hemostasis condition, the nasal packing 110 can be maintained in the nasal cavity NC applying pressure to the nasal tissue for a period of time. The hemostasis condition can then be reevaluated to determine if the target hemostasis condition has been met. Such a cycle can continue until the target hemostasis condition has been met, at which time the nasal packing 110 can be removed from the nasal cavity NC.

In some embodiments, as illustrated in FIG. 4D, prior to removal of the nasal packing 110 from the nasal tissue, pressure can be released, at 222, against the nasal tissue. For example, in the instance of a mechanical expander 120 being used to expand the nasal packing 110, a size or extent of the mechanical expander 120 can be reduced to reduce the pressure of the nasal packing 110 against the nasal tissue. As illustrated in FIG. 4E, the nasal packing 110 can be removed, at 224, from the nasal cavity NC.

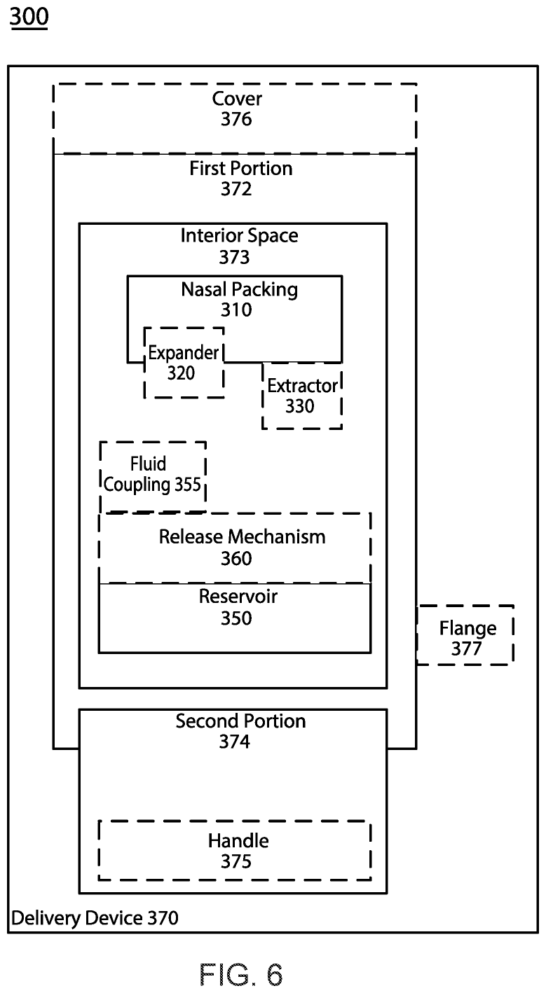

FIG. 6 is a schematic illustration of an epistaxis treatment system 300. The system 300 can be the same or similar in structure and/or function to any of the epistaxis treatment systems described herein. The system 300 incudes a delivery device 370, a nasal packing 310, and a reservoir 350. The delivery device 370 includes a first portion 372 (also referred to as a “first section”). The first portion 372 defines an interior space 373 within which the nasal packing 310 can be disposed. In some embodiments, the first portion 372 defines an open distal end such that the nasal packing 310 can move from the interior space 373 through the distal end during delivery of the nasal packing 310 from the first portion 372.

The first portion 372 can be formed in any suitable shape. For example, the first portion 372 can include a cylindrical housing and the interior space 373 defined by the housing can be cylindrical. In some embodiments, the first portion 372 can include a flange 377 (e.g., a circumferential flange) engageable by a user to improve grip during manipulation of the first portion 372. In some embodiments, rather than the flange 377, the first portion 372 can include a pair of opposing engagement portions shaped to be gripped by a user's fingers for manipulation of the first portion 372.

The delivery device 370 includes a second portion 374 (also referred to as a “second section”) partially extending into the interior space 373. The second portion 374 is configured to apply force to the nasal packing 310 such that the nasal packing 310 moves relative to the first portion 372 prior to and/or during delivery. In some embodiments, the second portion 374 can be advanced by a user (e.g., a patient or caregiver) to urge the nasal packing 310 through and out of the distal end of the first portion 372. For example, a distal end of the second portion 374 can contact a proximal end of the nasal packing 310 to urge the nasal packing 310 out of the distal end of the first portion 372 (e.g., prior to inserting the nasal packing 310 into the nasal cavity or after inserting a distal end of the first portion 372 into the nasal cavity). In some embodiments, with a distal end of the second portion 374 disposed in the interior space 373 of the first portion, the second portion 374 can be held stationary by a user and the first portion can be translated proximally relative to the second portion 374 while the nasal packing 310 is prevented from moving proximally by the distal end of the second portion such that the first portion 372 translated proximally relative to the nasal packing 310 and the nasal packing 310 is delivered from a distal end of the first portion 372. In some embodiments, a combination of the first portion 372 being moved proximally and the second portion 374 being moved distally can cause the nasal packing 310 to be delivered from the distal end of the first portion 372. In some embodiments, the second portion 374 can include a handle 375 to help the user grip, hold, and/or move the second portion 374. The handle 375 can include, for example, one or more flanges, hooks, and/or loops to assist the user in gripping the handle 375.

In some embodiments, the delivery device 370 includes an optional cover 376 configured to cover the distal end of the first portion 372 to enclose the interior space 373 prior to use of the delivery device 370 for delivery of the nasal packing 310. The cover 376 can be formed, for example, as a removable cap (e.g., attachable to the first portion 372 via a friction fit or threads) or as a film (e.g., a peelable film or a film that is breakable under pressure of the nasal packing 310 during delivery). In some embodiments, the cover 374 can be formed of a shrink wrap material cover the distal end of the delivery device 370 and positioned over at least a portion of the length of the first portion 372.

The nasal packing 310 can be the same or similar in structure and/or function to any of the nasal packing described herein. For example, the nasal packing 310 can include an optional expander 320 and/or an optional extractor 330. The expander 320 and the extractor 330 can be the same or similar in structure and/or function to any of the expanders or extractors, respectively, described herein.

As shown in FIG. 6, the reservoir 350 can be disposed in the interior space 373. The reservoir 350 can be configured to be displaced and/or pressurized such that medication within the reservoir 350 flows from the reservoir 350 to the nasal packing 310. For example, the reservoir 350 can optionally include a release mechanism 360 and/or a fluid coupling 355 that may be the same or similar in structure and/or function to any of the release mechanisms and/or fluid couplings described herein. In some embodiments, the fluid coupling 355 can be a portion of the interior space 373 defined by the housing of the first portion 372 via which medication can flow from the reservoir 350 to the nasal packing 310. In some embodiments, the fluid coupling 355 includes a separate tubular member defining a lumen through which medication can flow from the reservoir 350 to the nasal packing. In some embodiments, the tubular member can include only a proximal and distal opening. In some embodiments, the tubular member includes additional openings defined along a sidewall of the tubular member between the proximal and distal end such that the medication can flow from the tubular member to the nasal packing 310 via the various openings. In some embodiments, the release mechanism 360 can be coupled to the reservoir and configured to transition from a closed condition to an open condition to allow medication to flow from the reservoir 350 and to the nasal packing 310 via the fluid coupling 355 in response to a pressure above a threshold pressure within the reservoir 350 (e.g., applied by the distal end of the second portion 374). For example, in some embodiments, the release mechanism 360 can include a weakened or frangible portion of a sidewall of the reservoir 350. In some embodiments, the release mechanism 360 includes a valve that transitions from a closed to an open condition upon an internal pressure of the reservoir 350 rising above a threshold pressure. In some embodiments, the release mechanism 360 can be a frangible portion of the reservoir 350 configured to break when pushed against an internal obstruction (not shown) within the interior space 373 such that the medication within the reservoir 350 can be released from the reservoir 350. In some embodiments, the first portion 372 can include a filter (not shown) between the reservoir 350 and the nasal packing 310 to prevent unwanted material from reaching the nasal packing 110.

Prior to use, the delivery device 370 can be disposed in an initial configuration. In the initial configuration, the second portion 374 can be retracted relative to the first portion such that the nasal packing 310 and the reservoir 350 are disposed within the interior space 373. The nasal packing 310 can be disposed distally of the reservoir 350. The optional cover 376 can cover the distal opening of the first portion 372. To use the delivery device 370, the cover 376 can be optionally removed. The second portion 374 can then be translated relative to the first portion 372 (e.g., the second portion 374 can be advanced and/or the first portion 372 can be retracted) such that the medication within the reservoir flows to the nasal packing 310. The second portion 374 can then be further translated relative to the first portion 372 such that the nasal packing 310 is disposed within the orifice distally of the first portion 372. In some embodiments, the distal end of the first portion 372 can be disposed within an orifice of the user (e.g., the user's nasal cavity) prior to moving the nasal packing 310 to a position distal of the first portion 372 (e.g., within the nasal cavity). In some embodiments, the nasal packing 310 can be at least partially moved to a position distal of the first portion 372 outside of the nose and then the delivery device 370 can be used to insert the nasal packing 310 into the user's nose.

The nasal packing 310 can optionally be expanded against target tissue using the expander 320. The nasal packing 310 can be removed from the nasal packing 310 using the extractor 330. The expander 320 and the extractor 330 can be the same or similar in structure and/or function to any of the expanders and extractors, respectively, described herein.

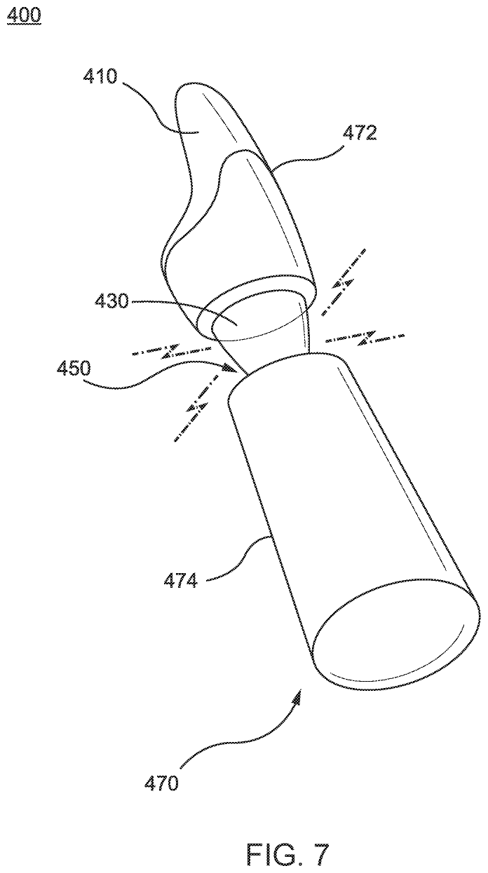

FIG. 7 is a schematic illustration of an epistaxis treatment system 400. The system 400 can be the same or similar in structure and/or function to any of the delivery or treatment systems described herein. For example, the system 400 can include a delivery device 470, a reservoir 450, and a nasal packing 410. The delivery device 470 can include a first portion 472 and a second portion 474. The second portion 474 can define or include the reservoir 450. The nasal packing 410 can extend distally of the first portion 472 and can include or be coupled to an extractor 430 on a proximal end of the nasal packing 410. The extractor 430 can, for example, form a proximal end of the nasal packing 410. The first portion 472 and the second portion 474 can be removably coupled via any suitable coupling mechanism, such as, for example, threads, a perforated portion, a weakened portion, and/or breakable tabs. Thus, the first portion 472 can be separated from the second portion 474 as shown in FIG. 7 (e.g., via unscrewing or breaking a connection between the first portion 472 and the second portion 474). The nasal packing 410 can then be wetted with the medication in the reservoir 450 of the second portion 474 (e.g., via the extractor 430 or via inverting the first portion and placing the distal end of the nasal packing 410 in contact with the medication in the reservoir 450).

In some embodiments, the second portion 474 can include a filter to prevent unwanted material (e.g., particles above a certain size and/or glass pieces) from reaching the nasal packing 410. In some embodiments, the second portion 474 includes a release mechanism configured to be transitioned from a closed condition to an open condition such that the medication in the reservoir 450 can be accessed. In some embodiments, the release mechanism can include a valve. In some embodiments, the release mechanism or the second portion 474 can include a removable or breakable covering or lid that contains the medication within the reservoir 450. The covering or lid can be removed or broken (e.g., as a result of the separation of the first portion 472 from the second portion 474 or by the user after the separation) such that the reservoir 450 is accessible by the nasal packing 410.

After wetting the nasal packing 410 with the medication within the second portion 474, the distal end of the nasal packing 410 can them be disposed within the orifice of the user (e.g., within the user's nasal cavity). In some embodiments, the second portion 474 can be deformable (e.g., squeezable) to urge the medication from the reservoir 450.

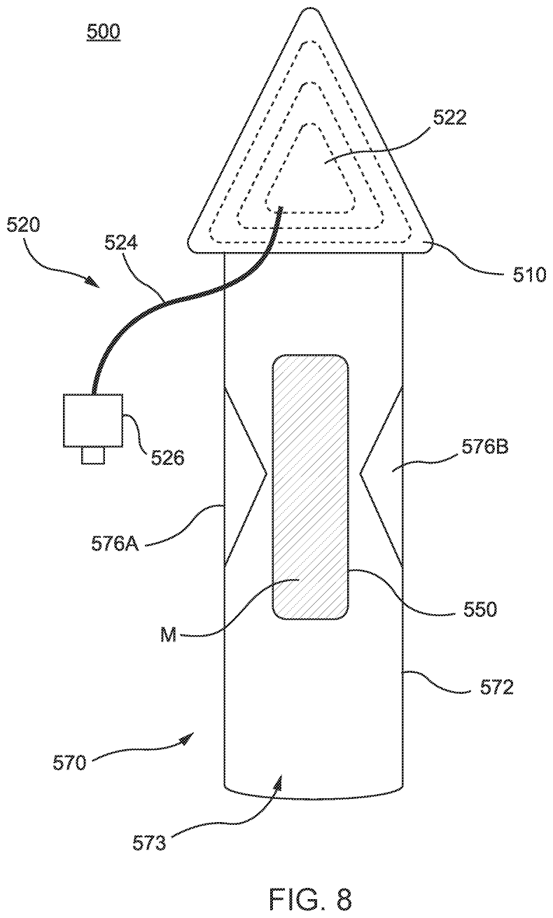

FIG. 8 is a schematic illustration of an epistaxis treatment system 500. The system 500 can be the same or similar in structure and/or function to any of the delivery or treatment systems described herein. For example, the system 500 can include a delivery device 570, a reservoir 550, and a nasal packing 510. The system 500 can also include an expander assembly 520 including an inflatable expander 522, a tubular member 524, and a source of fluid pressure 526. The delivery device 570 can include a tubular body 572 having at least one deformable sidewall portion. For example, the delivery device 570 can include a first deformable sidewall portion 576A and a second deformable sidewall portion 576B opposite the first deformable sidewall portion 576A. In some embodiments, the deformable sidewall portions are weakened and/or more flexible than a remainder of the tubular body 572 of the delivery device 570. The tubular body 572 defines an interior space 573 within which the reservoir 550 can be disposed. The reservoir 550 can be the same or similar in structure and/or function to any of the reservoirs described herein. For example, in some embodiments, the reservoir 550 can be formed as a bladder that bursts as a result of being deformed under the pressure of the first deformable sidewall portion 576A and/or the second deformable sidewall portion 576B. Thus, prior to or after insertion of the nasal packing 510 into an orifice of the user (e.g., the user's nasal cavity), the first deformable sidewall portion 576A and the second deformable sidewall portion 576B can be deformed toward the reservoir 550 to apply pressure to the reservoir 550 until the medication(s) flows from the reservoir 550 to the nasal packing 510 (e.g., due to the reservoir 550 bursting or a release mechanism associated with the reservoir 550 transitioning from a closed to an open condition). In some embodiments, the tubular body 572 can be used to deliver the nasal packing 510 into the nasal cavity. In some embodiments, the nasal packing 510 can be separated from the tubular body 572 after being wetted with medication(s) and inserted into the nasal cavity (e.g., by hand).

Prior to and/or after disposing the nasal packing 510 within the nasal cavity of the user, the expander assembly 520 can be used to inflate the inflatable expander 522 to increase the pressure of the nasal packing 510 against tissue defining the orifice of the user. For example, the source of fluid pressure 526 can include a bladder that can be compressed to push fluid from the bladder into the inflatable expander 522 via a lumen of the tubular member 524. To reduce the pressure applied by the inflatable expander 522 against the nasal packing 510, fluid can be drawn from the inflatable expander 522. For example, in embodiments in which the source of fluid pressure 526 is a bladder biased toward an expanded or inflated configuration, the pressure applied by the inflatable expander 522 can be reduced by allowing the bladder of the source of fluid pressure 526 to return to the expanded or inflated configuration).

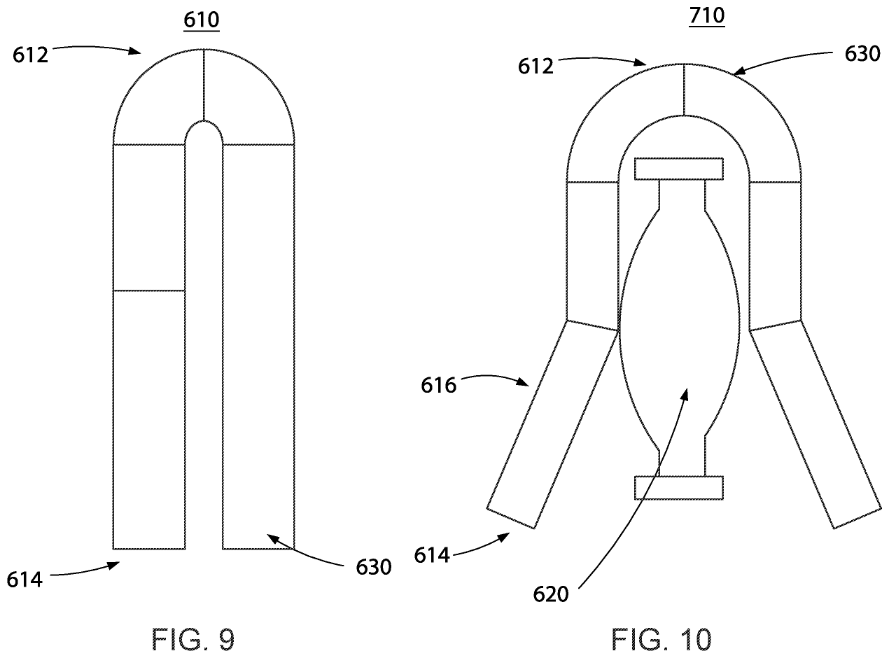

FIG. 9 is a schematic illustration of a nasal packing 610. The nasal packing 610 has a distal end 612 and a proximal end 614. In some embodiments, the nasal packing 610 can include and/or be coupled to an extractor 630, which may be formed as a portion of the proximal end 614 of the nasal packing 610. The nasal packing 610 can have a cylindrical shape or a U-shape in an unexpanded configuration. In some embodiments, the nasal packing 610 can be configured to expand upon being wetted with medication and/or upon being delivered from a constrained space within a delivery device. In some embodiments, the nasal packing 610 can be disposed on an end of an applicator to be wetted with medication, and then can be removed from the applicator and inserted into a nasal cavity (e.g., by hand).

As shown in FIG. 10, an expander 620 can be disposed within an interior of the nasal packing 610. The expander 620 can be, for example, a mechanical spring. Upon expanding the expander 620 from an unexpanded to an expanded configuration, the expander 620 can press the nasal packing 610 outward such that the nasal packing 610 has a larger outer diameter and/or such that the nasal packing 610 applies increased pressure to tissue surrounding the nasal packing 610. Additionally, the nasal packing 610 can optionally have a tapered portion 616 tapering away from an untapered distal portion. In some embodiments, the distal portion 612 can be inserted into the nasal cavity first. In some embodiments, the proximal portion 614 can be inserted into the nasal cavity first.

In some embodiments, the nasal packing 610 can be pre-soaked with medication(s) prior to being delivered to a nasal cavity. In some embodiments, a tubular member defining a lumen can extend from a reservoir toward, through, and/or into contact with the nasal packing 610 prior to and/or after delivery of the nasal packing 610 to a nasal cavity. The tubular member can define a set of sidewall openings such that medication(s) can flow from the reservoir toward various portions of the nasal packing via the sidewall openings.

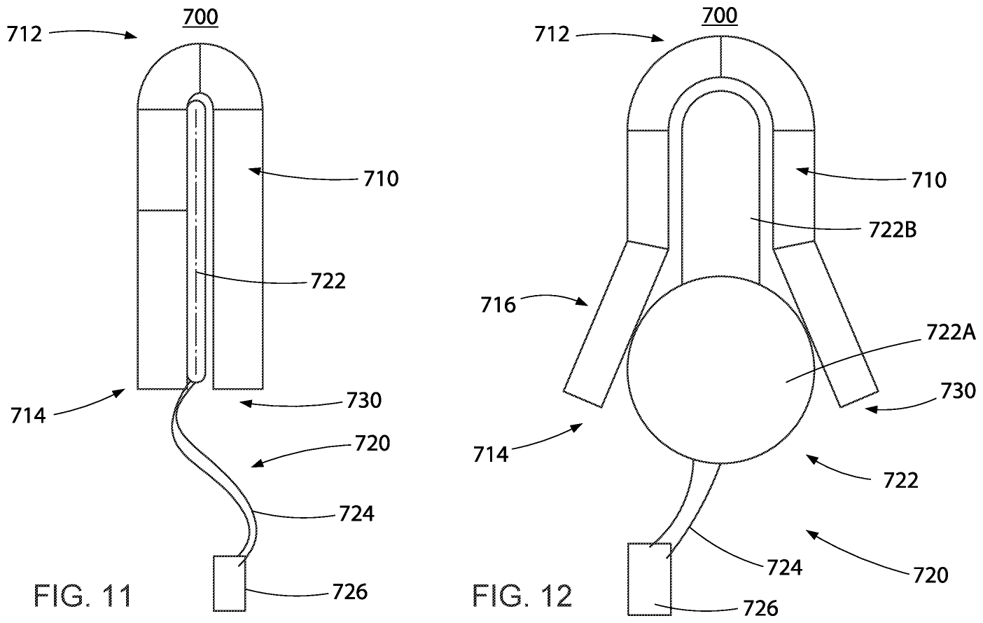

FIG. 11 is a schematic illustration of an epistaxis treatment system 700. The epistaxis treatment system 700 includes a nasal packing 710 and an expander assembly 720 including an inflatable expander 722, a tubular member 724, and a source of fluid pressure 726. The nasal packing 710 has a distal end 712 and a proximal end 714. In some embodiments, the nasal packing 710 can include and/or be coupled to an extractor 730, which may be formed as a portion of the proximal end 714 of the nasal packing 710. The nasal packing 610 can have a cylindrical shape or a U-shape in an unexpanded configuration.

As shown in FIG. 12, the inflatable expander 722 can be disposed within an interior of the nasal packing 710. Upon expanding the inflatable expander 722 from an unexpanded to an expanded configuration, the inflatable expander 722 can press the nasal packing 710 outward such that the nasal packing 710 has a larger outer diameter and/or such that the nasal packing 710 applies increased pressure to tissue surrounding the nasal packing 710. The inflatable expander 722 can include a first portion 722B (e.g., a cylindrical portion) and a second portion 722A (e.g., a spherical portion) having a wider diameter than the first portion 722B. In some embodiments, the nasal packing 710 can include a tapered portion 716 tapering away from an untapered distal portion. In some embodiments, the second portion 722A can expand to urge the portion 716 into a tapered configuration and/or to urge the portion 716 against surround nasal tissue. In some embodiments, the distal portion 712 can be inserted into the nasal cavity first. In some embodiments, the proximal portion 714 can be inserted into the nasal cavity first.

In some embodiments, similar to the nasal packing 610, the nasal packing 710 can be pre-soaked with medication(s) prior to being delivered to a nasal cavity. In some embodiments, a tubular member defining a lumen can extend from a reservoir toward, through, and/or into contact with the nasal packing 710 prior to and/or after delivery of the nasal packing 710 to a nasal cavity. The tubular member can define a set of sidewall openings such that medication can flow from the reservoir toward various portions of the nasal packing via the sidewall openings.

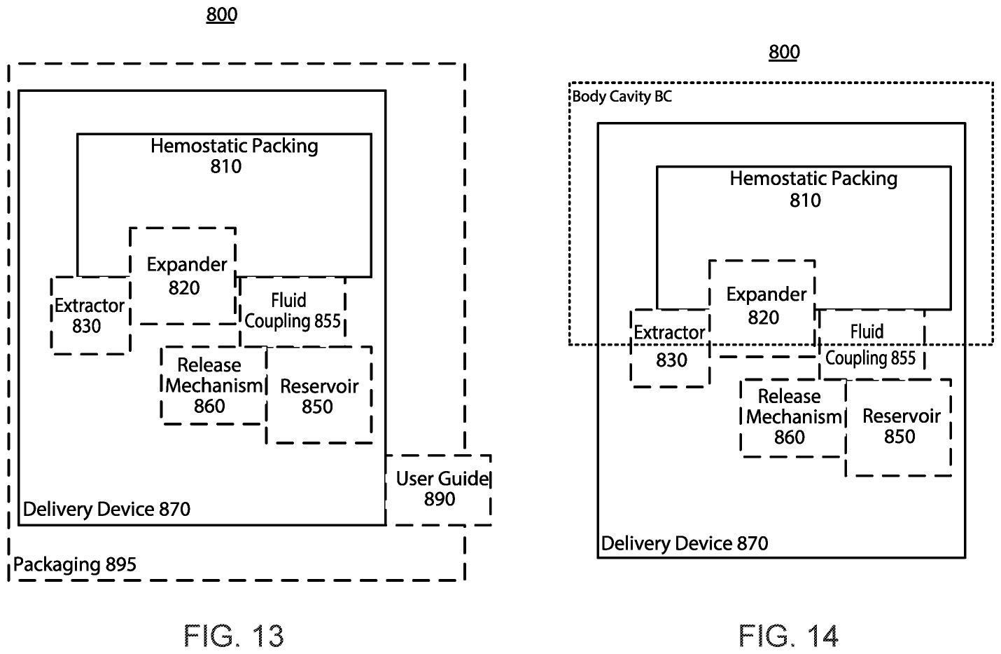

While the epistaxis treatment systems are described herein as being used for treatment of epistaxis within the nasal cavity, it should be understood that they have been presented by way of example only and not limitation. The embodiments and/or devices described herein are not intended to be limited to any specific implementation unless expressly stated otherwise. For example, the embodiments described herein can be used to stop bleeding in any natural or surgically-created cavities of a patient. For example, the embodiments described herein can be used to stop bleeding in any suitable natural or surgically-created cavities or lumens, or in any organs or spaces accessible by natural or surgically-created orifices and/or lumens. For example, the embodiments described herein can be used to address and stop bleeding in uterine, vaginal, urethral, bladder, rectal, colon, esophageal, bronchial, oral, or any other suitable applications. For example, FIG. 13 is a schematic illustration of a treatment device 800 used to stop bleeding in a body cavity or lumen. The treatment device 800 can be the same or similar in structure and/or function to any of the treatment devices described herein, such as the treatment device 100. For example, the treatment device 800 includes a hemostatic packing 810, an optional expander 820 coupled to the hemostatic packing 810, an optional extractor 830 coupled to hemostatic packing 810, one or more optional reservoirs 850 fluidically coupled to the hemostatic packing 810 via a fluid coupling 855 and containing one or more medications, an optional release mechanism 860 coupled to the reservoir 850 and the fluid coupling 855, a delivery device 870 releasably coupleable to the hemostatic packing 810, an optional user guide 890 associated with the delivery device 870, and optional packaging 895 to contain the other components of the epistaxis treatment system 800. As shown in FIG. 14, after removal of the packaging 895, the delivery device 870 can be used to deliver the hemostatic packing 810 to a body cavity or lumen BC.

While various embodiments have been described herein, textually and/or graphically, it should be understood that they have been presented by way of example only, and not limitation. Likewise, it should be understood that the specific terminology used herein is for the purpose of describing particular embodiments and/or features or components thereof and is not intended to be limiting. Various modifications, changes, enhancements, and/or variations in form and/or detail may be made without departing from the scope of the disclosure and/or without altering the function and/or advantages thereof unless expressly stated otherwise. Functionally equivalent embodiments, implementations, and/or methods, in addition to those enumerated herein, will be apparent to those skilled in the art from the foregoing descriptions and are intended to fall within the scope of the disclosure.

Where schematics, embodiments, and/or implementations described above indicate certain components arranged and/or configured in certain orientations or positions, the arrangement of components may be modified, adjusted, optimized, etc. The specific size and/or specific shape of the various components can be different from the embodiments shown and/or can be otherwise modified, while still providing the functions as described herein. More specifically, the size and shape of the various components can be specifically selected for a desired or intended usage. Thus, it should be understood that the size, shape, and/or arrangement of the embodiments and/or components thereof can be adapted for a given use unless the context explicitly states otherwise. By way of example, in some implementations, a treatment device intended to provide treatment to an adult user may have a first size and/or shape, while a treatment device intended to provide treatment to a pediatric user may have a second size and/or shape smaller than the first size and/or shape. Moreover, the smaller size and/or shape of, for example, a pediatric treatment device may result in certain components being moved, reoriented, and/or rearranged while maintaining the desired function of the device.

Although various embodiments have been described as having particular characteristics, functions, components, elements, and/or features, other embodiments are possible having any combination and/or sub-combination of the characteristics, functions, components, elements, and/or features from any of the embodiments described herein, except mutually exclusive combinations or when clearly stated otherwise. Moreover, unless otherwise clearly indicated herein, any particular combination of components, functions, features, elements, etc. can be separated and/or segregated into independent components, functions, features, elements, etc. or can integrated into a single or unitary component, function, feature, element, etc.

Where methods described above indicate certain events occurring in certain order, the ordering of certain events may be modified. Additionally, certain of the events may be performed concurrently in a parallel process when possible, as well as performed sequentially as described above. While methods have been described as having particular steps and/or combinations of steps, other methods are possible having a combination of any steps from any of methods described herein, except mutually exclusive combinations and/or unless the context clearly states otherwise.

1. A system, comprising:
a nasal packing;
a delivery device releasably couplable to the nasal packing and configured to deliver the nasal packing into a nasal cavity of a subject;
a reservoir configured to contain medication; and
a fluid coupling configured such that the medication can flow from the reservoir to the nasal packing via the fluid coupling.

2. The system of claim 1, wherein the delivery device includes a first portion and a second portion, the first portion configured to be moved relative to the second portion such that the nasal packing is disposed distally of a distal end of the delivery device.

3. The system of claim 1, further comprising an extractor having a first end coupled to the nasal packing and a free second end configured to be grasped when the nasal packing is disposed in the nasal cavity and pulled to withdraw the nasal packing from the nasal cavity.

4. The system of claim 1, wherein the nasal packing includes a proximal portion formed as an extractor configured to be grasped when the nasal packing is disposed in the nasal cavity and pulled to withdraw the nasal packing from the nasal cavity.

5. The system of claim 1, wherein the reservoir contains the medication.

6. The system of claim 5, wherein the medication includes at least one of a vasoconstrictor or an antifibrinolytic.

7-10. (canceled)

11. The system of claim 1, wherein the nasal packing includes a base layer and an outer layer formed of a different material than the base layer.

12. The system of claim 1, further comprising a release mechanism coupled to the reservoir and configured to transition from a closed condition to an open condition to allow medication to flow from the reservoir and to the nasal packing via the fluid coupling in response to a pressure above a threshold pressure within the reservoir.

13-14. (canceled)

15. The system of claim 1, wherein the nasal packing is configured to at least one of expand or be expanded upon delivery from the delivery device into the nasal cavity.

16. A system, comprising:
a nasal packing;
a delivery device releasably couplable to the nasal packing and configured to deliver the nasal packing into a nasal cavity of a subject; and
an expander configured to cause the nasal packing to expand in the nasal cavity to apply pressure against nasal tissue at least partially defining the nasal cavity.

17. The system of claim 16, wherein the expander is coupled to the nasal packing and configured to be delivered to the nasal cavity by the delivery device.

18. The system of claim 17, wherein the expander is biased toward an expanded configuration such that the expander is configured to self-expand upon delivery of the nasal packing into the nasal cavity.

19-24. (canceled)

25. The system of claim 16, wherein the nasal packing defines a reservoir configured to contain medication prior to delivery of the nasal packing to the nasal cavity.

26. The system of claim 16, further comprising a reservoir configured to contain medication and a fluid coupling configured such that the medication can flow from the reservoir to the nasal packing via the fluid coupling.

27. A method, comprising:
delivering a nasal packing to a nasal cavity;
allowing a release of medication from the nasal packing to nasal tissue at least partially defining the nasal cavity;
maintaining the nasal packing in the nasal cavity such that the nasal packing applies pressure against the nasal tissue;
evaluating whether a hemostasis condition of the nasal tissue meets a hemostasis target; and
removing, in response to the hemostasis condition meeting the hemostasis target, the nasal packing from the nasal cavity.

28. The method of claim 27, wherein delivering the nasal packing to the nasal cavity includes:
disposing a distal end of a delivery device within the nasal cavity with the nasal packing releasably coupled to the delivery device; and
moving a first portion of the delivery device relative to a second portion of the delivery device such that the nasal packing is disposed distally of the distal end of the delivery device.

29-31. (canceled)

32. The method of claim 27, further comprising:
prior to allowing the release of medication, pressing the nasal packing against nasal tissue at least partially defining the nasal cavity.

33. The method of claim 27, further comprising:
in response to the hemostasis condition not meeting the hemostasis target, continuing to maintain the nasal packing in the nasal cavity such that the nasal packing applies pressure against the nasal tissue; and
reevaluating whether the hemostasis condition of the nasal tissue meets the hemostasis target.

34-40. (canceled)

41. A system, comprising:
a hemostatic packing;
a delivery device releasably couplable to the hemostatic packing and configured to deliver the hemostatic packing into a body cavity of a subject;
a reservoir configured to contain medication; and
a fluid coupling configured such that the medication can flow from the reservoir to the hemostatic packing via the fluid coupling.

42. The system of claim 41, wherein the hemostatic packing is configured to at least one of expand or be expanded upon delivery from the delivery device into the body cavity.

43. (canceled)
